(19) United States
(12) Patent Application Publication
(10) Pub. No.: US 2024/0389872 A1
(43) Pub. Date: Nov. 28, 2024

(54) DYNAMIC VOLTAGE SCALING (DVS) IN PHOTOPLETHYSMOGRAPHY (PPG) SYSTEMS

(71) Applicant: Analog Devices International Unlimited Company, Limerick (IE)

(72) Inventors: Yaohua YANG, West Linn, OR (US); Nutan Prasad, Saratoga, CA (US); Yi Zhang, Beaverton, OR (US); Manuel Di Cera, Nova Siri (IT); Daniel Melis, Oristano (IT); Marco Masini, Milano (IT); Elio Consoli, Gravina di Catania (IT); Giuseppe Patti, Catania (IT); Angelo Mazzone, Ornago (IT); Forrest Christo, Bend, OR (US); Jeremy Georges, Colorado Springs, CO (US); Craig Easson, San Jose, CA (US)

(21) Appl. No.: 18/627,153

(22) Filed: Apr. 4, 2024

Related U.S. Application Data
(60) Provisional application No. 63/468,723, filed on May 24, 2023.

(51) Int. Cl. A61B 5/024

(52) U.S. Cl. CPC A61B 5/02427; A61B 2560/0223

(57) ABSTRACT

Aspects of the disclosure relate to a device that includes a plurality of light sources comprising a first light source and a second light source configured to emit electromagnetic radiation. The device may be configured to determine to perform a first set of measurements on the subject within a time window, wherein the first set of measurements comprise a first measurement performed with the first light source using a first optimal voltage and a second measurement performed with the second light source using a second optimal voltage, wherein the first measurement and the second measurement are performed in a contiguous sequence within the time window, and wherein the first optimal voltage is different from the second optimal voltage. The device may be further configured to apply the first optimal voltage to the first light source and apply the second optimal voltage to the second light source.

### CROSS REFERENCE TO RELATED APPLICATION(S)

This application claims the benefit of U.S. Provisional Application Ser. No. 63/468,723, entitled “DYNAMIC VOLTAGE SCALING (DVS) FOR LED POWER SUPPLIES IN PPG APPLICATIONS” and filed on May 24, 2023, the disclosure of which is expressly incorporated by reference herein in its entirety.

### BACKGROUND

Photoplethysmography (PPG) relates to optical techniques used to detect volumetric changes in blood circulation and other health related parameters of a subject. PPG is implemented in various systems such as pulse oximeter sensors, and provides a low cost and non-invasive technique for making measurements at the surface of the skin. Typical PPG systems may include a device having a light source (e.g., light emitting diode (LED)) and a photodetector. The light source radiates light on skin tissue and the photodetector measures light reflected from the tissue. The relative intensities of the reflected light received by the photodetector, and how those intensities vary in response to the heartbeat pulse, provide a measure of blood oxygenation level. In one example, measurements of the reflected light may indicate a percentage oxygen saturation in arterial hemoglobin, which indicates percentage of hemoglobin molecules in the arteries that contain an oxygen molecule. This measurement is commonly referred to as saturation of peripheral oxygen, or SpO2.

Oxygenated and deoxygenated blood have different absorption levels for different wavelengths. For example, green, red, infrared (IR) light, and others may be used to measure oxygen absorption levels in blood. FIG. 1 is a line graph illustrating a difference in absorbance levels of two different light wavelengths in the red band (660 nanometers (nm)) and IR band (910 nm), by oxygenated hemoglobin (HbO2) and deoxygenated hemoglobin (Hb). The ratio of the absorbance of HbO2 to Hb is lower in the red portion of the spectrum than it is in the IR portion of the spectrum. In other words, Hb absorbs red light more readily than HbO2, and HbO2 absorbs IR light more readily than Hb.

Power used by the light sources on a PPG system makes up a significant portion of the power consumed by such systems. To the extent many PPG systems include relatively small, wearable devices that are battery powered, there exists a need for further improvements in power management. Such improvements may also be applicable to other technologies and standards that employ these technologies.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

Aspects are directed to a method for capturing measurements related to health of a subject. In some examples, the method includes determining to perform a first set of measurements on the subject within a time window, wherein the first set of measurements comprise a first measurement performed with a first light source using a first optimal voltage and a second measurement performed with a second light source using a second optimal voltage, wherein the first measurement and the second measurement are performed in a contiguous sequence within the time window, and wherein the first optimal voltage is different from the second optimal voltage. In some examples, the method includes applying the first optimal voltage to the first light source. In some examples, the method includes applying the second optimal voltage to the second light source.

Aspects are directed to a method for capturing measurements related to health of a subject at an analog front end (AFE). In some examples, the method includes receiving, from a microcontroller unit (MCU) via a first interface, a measurement configuration including an indication of a first set of measurements that comprise a first measurement performed with a first light source using a first optimal voltage and a second measurement performed with a second light source using a second optimal voltage. In some examples, the method includes transmitting, to a buck boost converter via a second interface, a first command configured to cause the buck boost to apply the first optimal voltage to the first light source. In some examples, the method includes transmitting, to the buck boost converter via the second interface, a second command configured to cause the buck boost to apply the second optimal voltage to the second light source.

Aspects are directed to an apparatus for capturing measurements related to health of a subject. In some examples, the apparatus includes means for determining to perform a first set of measurements on the subject within a time window, wherein the first set of measurements comprise a first measurement performed with a first light source using a first optimal voltage and a second measurement performed with a second light source using a second optimal voltage, wherein the first measurement and the second measurement are performed in a contiguous sequence within the time window, and wherein the first optimal voltage is different from the second optimal voltage. In some examples, the apparatus includes means for applying the first optimal voltage to the first light source. In some examples, the apparatus includes means for applying the second optimal voltage to the second light source.

Aspects are directed to an apparatus for capturing measurements related to health of a subject. In some examples, the apparatus includes means for receiving, from a microcontroller unit (MCU) via a first interface, a measurement configuration including an indication of a first set of measurements that comprise a first measurement performed with a first light source using a first optimal voltage and a second measurement performed with a second light source using a second optimal voltage. In some examples, the apparatus includes means for transmitting, to a buck boost converter via a second interface, a first command configured to cause the buck boost to apply the first optimal voltage to the first light source. In some examples, the apparatus includes means for transmitting, to the buck boost converter via the second interface, a second command configured to cause the buck boost to apply the second optimal voltage to the second light source.

Aspects are directed to an apparatus for capturing measurements related to health of a subject. The apparatus includes one or more memories, individually or in combination, having instructions, and one or more processors, individually or in combination, configured to execute the instructions. In some examples, the one or more processors are configured to determine to perform a first set of measurements on the subject within a time window, wherein the first set of measurements comprise a first measurement performed with a first light source using a first optimal voltage and a second measurement performed with a second light source using a second optimal voltage, wherein the first measurement and the second measurement are performed in a contiguous sequence within the time window, and wherein the first optimal voltage is different from the second optimal voltage. In some examples, the one or more processors are configured to apply the first optimal voltage to the first light source. In some examples, the one or more processors are configured to apply the second optimal voltage to the second light source.

Aspects are directed to an apparatus for capturing measurements related to health of a subject. The apparatus includes one or more memories, individually or in combination, having instructions, and one or more processors, individually or in combination, configured to execute the instructions. In some examples, the one or more processors are configured to receive, from a microcontroller unit (MCU) via a first interface, a measurement configuration including an indication of a first set of measurements that comprise a first measurement performed with a first light source using a first optimal voltage and a second measurement performed with a second light source using a second optimal voltage. In some examples, the one or more processors are configured to transmit, to a buck boost converter via a second interface, a first command configured to cause the buck boost to apply the first optimal voltage to the first light source. In some examples, the one or more processors are configured to transmit, to the buck boost converter via the second interface, a second command configured to cause the buck boost to apply the second optimal voltage to the second light source.

Aspects are directed to non-transitory computer-readable medium comprising instructions that, when executed by an apparatus, cause the apparatus to perform operations. In some examples, the operations include determining to perform a first set of measurements on the subject within a time window, wherein the first set of measurements comprise a first measurement performed with a first light source using a first optimal voltage and a second measurement performed with a second light source using a second optimal voltage, wherein the first measurement and the second measurement are performed in a contiguous sequence within the time window, and wherein the first optimal voltage is different from the second optimal voltage. In some examples, the operations include applying the first optimal voltage to the first light source. In some examples, the operations include applying the second optimal voltage to the second light source.

Aspects are directed to non-transitory computer-readable medium comprising instructions that, when executed by an apparatus, cause the apparatus to perform operations. In some examples, the operations include receiving, from a microcontroller unit (MCU) via a first interface, a measurement configuration including an indication of a first set of measurements that comprise a first measurement performed with a first light source using a first optimal voltage and a second measurement performed with a second light source using a second optimal voltage. In some examples, the operations include transmitting, to a buck boost converter via a second interface, a first command configured to cause the buck boost to apply the first optimal voltage to the first light source. In some examples, the operations include transmitting, to the buck boost converter via the second interface, a second command configured to cause the buck boost to apply the second optimal voltage to the second light source.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

The disclosed aspects will hereinafter be described in conjunction with the appended drawings, provided to illustrate and not to limit the disclosed aspects, wherein like designations denote like elements, and in which:

FIG. 1 is a line graph illustrating a difference in absorbance levels of two different light wavelengths in the red band and IR band by oxygenated hemoglobin (HbO2) and deoxygenated hemoglobin (Hb).

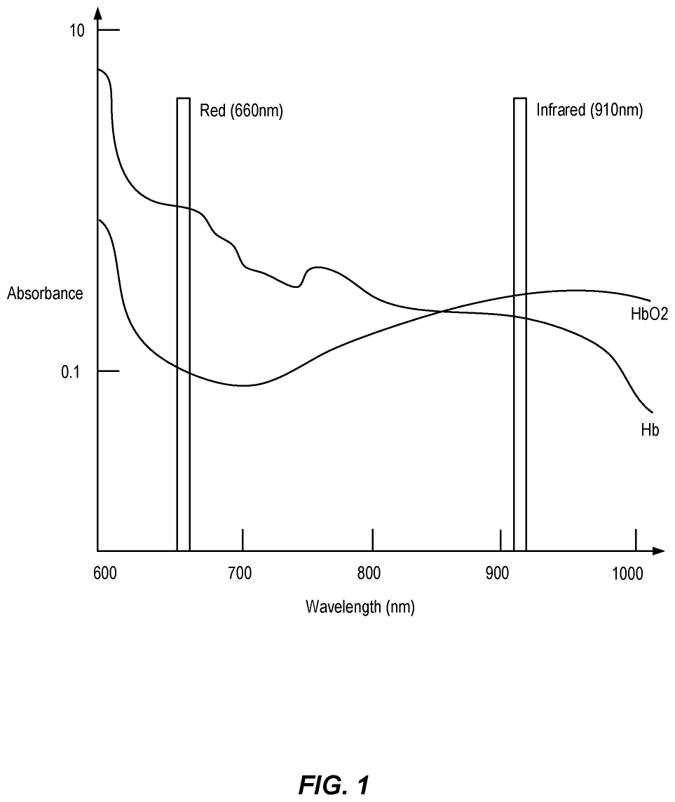

FIG. 2 is a schematic drawing illustrating an example photoplethysmography (PPG) device configured with dynamic voltage scaling (DVS).

FIG. 3 is a bar graph of LED voltage versus time frames for multiple LEDs, illustrating an example application of the DVS function of the PPG device of FIG. 2.

FIG. 4 is a block diagram illustrating an example of a PPG device configured for controlling a voltage applied across one or more light emitting diodes (LEDs) on a per-measurement basis.

FIG. 5 is a bar graph of LED voltage versus time frames for multiple LEDs, illustrating an example application of a fast DVS function of the PPG device of FIG. 4.

FIG. 6 is a flow diagram illustrating an example learning process for performing fast DVS (e.g., as illustrated in FIG. 5) at a PPG device (e.g., PPG device of FIG. 4).

FIG. 7 is a digital signal diagram illustrating example signaling between an analog front end (AFE) and a buck boost of a PPG device.

FIG. 8 is a block diagram illustrating an example look-up table that includes multiple rows, each corresponding to an optimal voltage for a particular measurement.

FIG. 9 is a simplified schematic illustrating a multipath nested Miller compensation (MNMC) op-amp that may be used to detect voltage dropout across the current sink.

FIG. 10 is a simplified flow diagram illustrating example operations that may be performed by a PPG device.

FIG. 11 is a simplified flow diagram illustrating example operations associated with a first function of FIG. 10.

FIG. 12 is a simplified flow diagram illustrating example operations that may be performed by an AFE of a PPG device.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Photoplethysmography (PPG) is an optical technique used to detect volumetric changes in blood in peripheral circulation. Through PPG, various measurements can be made, including heartrate and oxygen saturation. Conventional PPG systems consume a rather significant amount of power, which in some cases may reduce the life of a battery-powered PPG device. One of the reasons for the significant power consumption relates to the wide variation in the voltages required at the anode of the transmit light-emitting diode (LED). Conventional PPG systems use a static power supply for this voltage, leading to wasted power in the LED current sink. For example, a green LED may require 3.5V to operate, while an infrared (IR) LED may only require 1.5V, yet a conventional PPG system may apply the same voltage level across both the green LED and the IR LED.

Thus, aspects of the disclosure are directed to optimizing the LED power supply by dynamically adjusting the voltage across each LED based on which LED is being powered. For example, the LED power supply may apply 3.5V across the green LED, then apply 1.5V across the IR LED. By dynamically adjusting the voltage across the LEDs, power consumption of the PPG system may be reduced and the battery life extended.

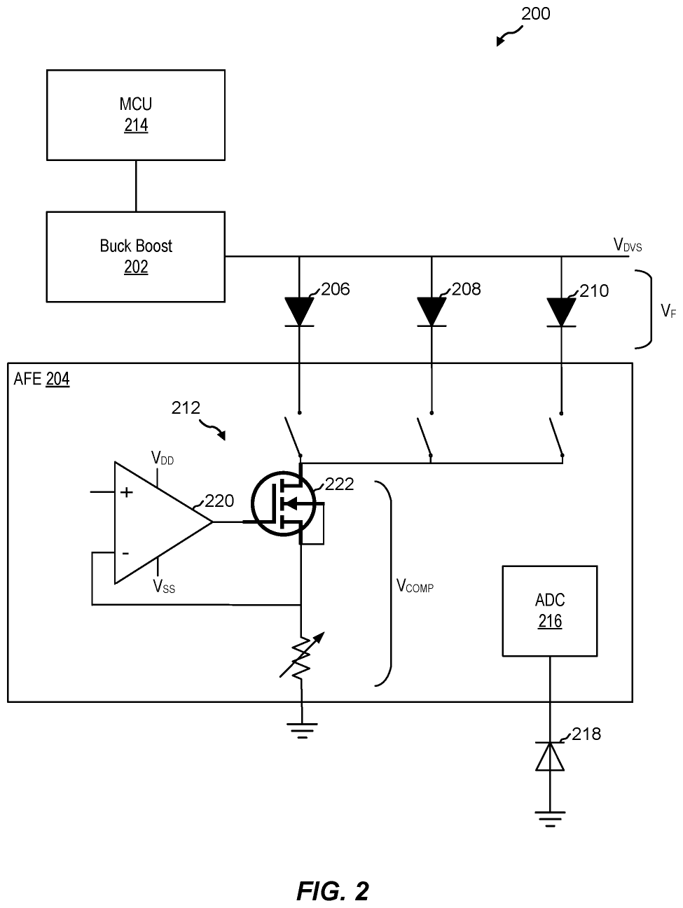

FIG. 2 is a schematic drawing illustrating an example photoplethysmography (PPG) device 200 configured with dynamic voltage scaling (DVS), of which one or more aspects of the present disclosure may utilize. As illustrated, the PPG device 200 includes a microcontroller unit (MCU) 214, a buck boost 202, an analog front end (AFE) 204, and multiple light emitting diodes (LEDs) (e.g., a first LED 206, a second LED 208, and a third LED 210). It should be noted that FIG. 2 illustrates a example PPG device 200, and that the features described herein are not limited to the illustrated circuit structure, but rather may be used with any suitable PPG circuit structure.

The MCU 214 may control aspects of buck boost functionality, such as controlling a voltage applied across one or more LEDs on a per-frame basis. The AFE 204 includes a current sink 212 configured to receive a current passed from the buck boost 202 through one or more of the of the LEDs. The current sink 212 includes an operational amplifier 220 (op amp) having an output coupled to the gate of a MOSFET 222 and a power supply (e.g., V_{DD}) and V_{SS}). One end of each LED is coupled to the buck boost 202, while another end of each LED is coupled to a drain of the MOSFET 222. The buck boost 202 may be configured with a DVS function that operates to vary the forward voltage applied across the LEDS (V_{F}) based on various parameters. For example, V_{F} may vary with the wavelength of light emitted from each LED and the current used by the corresponding LED. A current sink compliance voltage (V_{COMP}) is related to a minimum voltage that the current sink 212 needs for the AFE 204 to properly operate, and V_{COMP} may vary with the LED current. As such, the optimal V_{DVS} may be equal to the sum of V_{F} and V_{COMP} for a given LED. Any additional V_{DVS} is wasted as heat in the current sink 212.

In some examples, the AFE 204 further includes an analog-to-digital converter (ADC) 216 and a photodiode 218 configured to measure one or more of ambient light and/or light from one or more LEDs reflected from a subject. The ADC 216 may be used to convert a received intensity of the reflected light into a digital signal indicative of the received intensity.

The PPG device 200 may be configured to acquire a volumetric organ measurement by optical means. In one example, an LED may radiate light toward tissue of the subject (e.g., a human finger). A portion of the radiated light is absorbed by the finger and another portion of the radiated light is reflected by the finger. The reflected light may be observed and measured by the photodiode 218. The light observed and measured by photodiode 218 is a function of the amount of light absorbed by the tissue. Thus, the observed light may be used to derive information about the tissue.

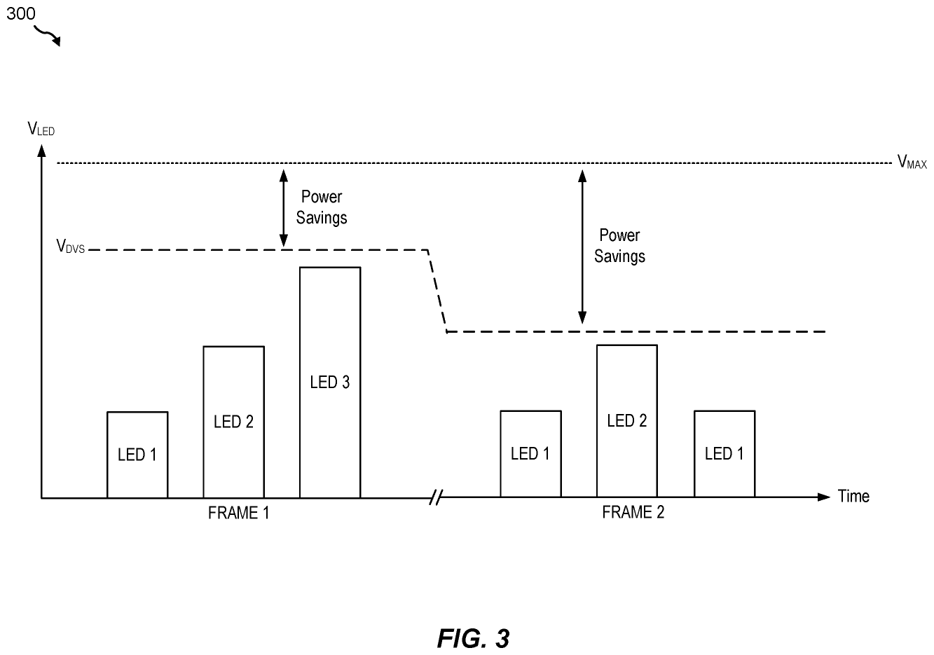

FIG. 3 is a bar graph 300 illustrating an example application of the DVS function of the PPG device 200 of FIG. 2. Here, at each frame, the buck boost 202 applies a constant voltage across the LEDs for the duration of the corresponding frame, but the voltage at each frame may vary relative to another frame based on which measurements are being performed within each frame. For example, using the DVS function, the buck boost 202 may control the voltage level across the LEDs based on the maximum voltage required to power the LEDs in a given frame.

PPG devices may segment measurements into time frames or time windows (e.g., illustrated as FRAME 1 and FRAME 2) to accurately capture and analyze measurement variations over time. A PPG frame rate may relate to a number of measurements or frames taken per unit of time (e.g., per second). The appropriate frame rate may depend on the specific application of the PPG device. For instance, when monitoring heart rate, a frame rate of 30 to 60 frames per second may be sufficient to capture the relatively slow changes in blood volume associated with each heartbeat. For more rapid physiological events, a higher frame rate might be necessary. Moreover, multiple measurements may be performed within a frame, with each measurement comprising multiple exposures (e.g., 10-100 μs) within a single measurement. Each exposure may relate to a flash of light from a light source, and an average value may be calculated based on information collected with each exposure within a single measurement.

In some examples, the rate of a light source (e.g., exposure rate of an LED) in a PPG device may relate to how often the light from the LED is shone onto the tissue of a subject during a single measurement (e.g., LED 1 of FRAME 1). This rate may be synchronized with the frame rate or sampling rate of the PPG device. In other words, the light source may be pulsed or modulated at the same frequency as the rate at which data is being collected. The frequency or rate of LED light exposure may vary depending on the device and application and may be in the range of tens to hundreds of Hertz (e.g., cycles per second). For example, a PPG device used for heart rate monitoring may pulse the LED at a frequency of 60-100 Hz, corresponding to a data collection rate of 60-100 samples per second during each measurement.

As illustrated, the PPG device 200 performs three measurements within a first frame (FRAME 1) using the first LED 206, the second LED 208, and the third LED 210. During a subsequent second frame (FRAME 2), the PPG device 200 may perform three measurements using the first LED 206 and the second LED 208. Here, the maximum V_{DVS} of the first frame is defined by the voltage required to power the third LED, whereas the maximum V_{DVS} of the second frame is defined by the voltage required to power the second LED. As such, the buck boost 202 may dynamically scale V_{DVS} per frame, thereby reducing the amount of power used by the PPG device 200 to perform measurements. As illustrated, V_{MAX} represents a maximum voltage that the buck boost may apply across the LEDs, and a delta between V_{MAX} and V_{DVS} at each frame shows a power savings relative to V_{MAX}.

Thus, while the MCU 214 may control the buck boost 202 as well as its DVS function to reduce power consumption of the PPG device 200, additional power may be conserved at the PPG device 200 if the LED voltage levels are controlled on a per-measurement basis. For example, the V_{DVS} of the first frame provides far more power to the first LED and the second LED than is needed to power them. This surplus power is dissipated as heat at the current sink 212, and may reduce battery life of the PPG device 200.

### Examples of Per-Measurement Voltage Scaling

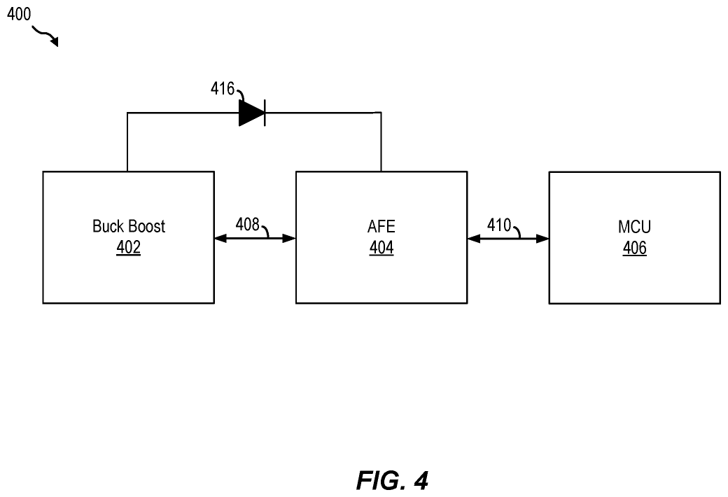

FIG. 4 is a block diagram illustrating an example of a PPG device 400 configured for controlling a voltage applied across one or more LEDs on a per-measurement basis. This contrasts with the other PPG device 200 illustrated in FIGS. 2 and 3, which is configured to control voltage applied across one or more LEDs on a per-frame basis. Moreover, in contrast with the other PPG device 200 that includes an MCU 214 having direct control of a buck boost 202, the PPG device 400 of FIG. 4 may include an MCU 406 coupled to an AFE 404 (e.g., AFE 204 of FIG. 2), where the AFE 404 is in direct control of the buck boost 402. Here, the AFE 404 controls the buck boost 402 because voltage level transitions occur per measurement, which is a relatively faster rate of transition compared to a per frame transition and the MCU 406 may not be capable of handling such a rapid transition. In other words, the PPG device 200 configuration of FIG. 2 may prevent per measurement voltage level transitions. One or more LEDs 416 may be coupled between the buck boost 402 and the AFE 404. It should be noted that the PPG device 400 may use the same or similar components illustrated in FIG. 2 (e.g., the buck boost 402 of FIG. 4 may be the same or similar to the buck boost 202 of FIG. 2).

In various examples, the PPG device 200 may be implemented as a chip, a system on a chip (SoC), or a device that may include: one or more processors, processing blocks or processing elements (collectively “processor”), and one or more memories or memory blocks (collectively “memory”).

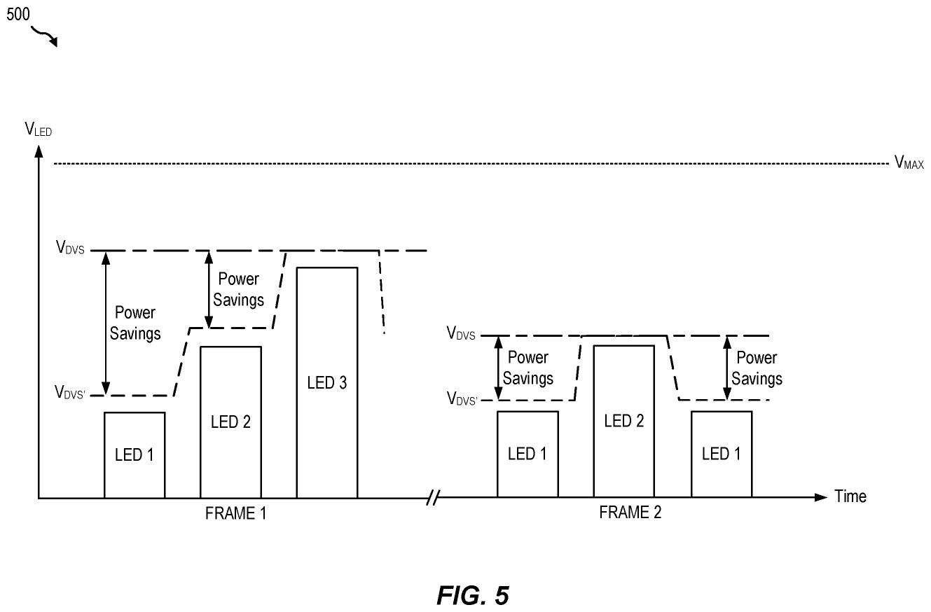

FIG. 5 is a bar graph 500 illustrating an example application of a fast DVS function of the PPG device 400 of FIG. 4. As illustrated, the bar graph 500 includes two frames: frame 1 and frame 2, both shown on corresponding portions of the x-axis. Each of the frames may include three measurements, with each measurement using a particular LED at a particular time. Here, instead of the buck boost applying a constant voltage to the LEDs for the duration of each frame, the buck boost 402 may adjust the voltage applied to the LEDs for each measurement. For example, using the fast DVS function, the buck boost 402 may control the voltage level across the LEDs based on the voltage needs of whichever LED is used for measuring at a given time.

As illustrated, the PPG device 400 performs three measurements within a first frame (FRAME 1) using a first LED (LED 1), a second LED (LED 2), and a third LED (LED 3). Here, the voltage applied (V_{DVS′}) to the first LED for the first measurement may be lower than voltages applied during the second and third measurements because the first LED may not require as much voltage as the second and third LEDs. Similarly, the voltage applied (V_{DVS′}) to the second LED for the second measurement may be lower than voltage applied to the third LED during the third measurement because the second LED may not require as much voltage as the third LED. Thus, the voltages applied at each measurement may be based on how much voltage is required to power on a given one or more LEDs during a corresponding measurement.

FIG. 5 also illustrates a delta voltage between the V_{DVS} of FIG. 3 and the V_{DVS′} of per-measurement, fast DVS function. There is significant power savings in the first frame of FIG. 5 because the PPG device 400 does not use the static V_{DVS}, but rather a dynamic V_{DVS′} based on per-measurement LED power needs. Similarly, power savings are also realized in the second frame because the first and third measurements (e.g., measurements using the first LED) do not require as high a level of voltage as the second LED in the second measurement.

### Examples of an AFE Configured with a Learning Algorithm

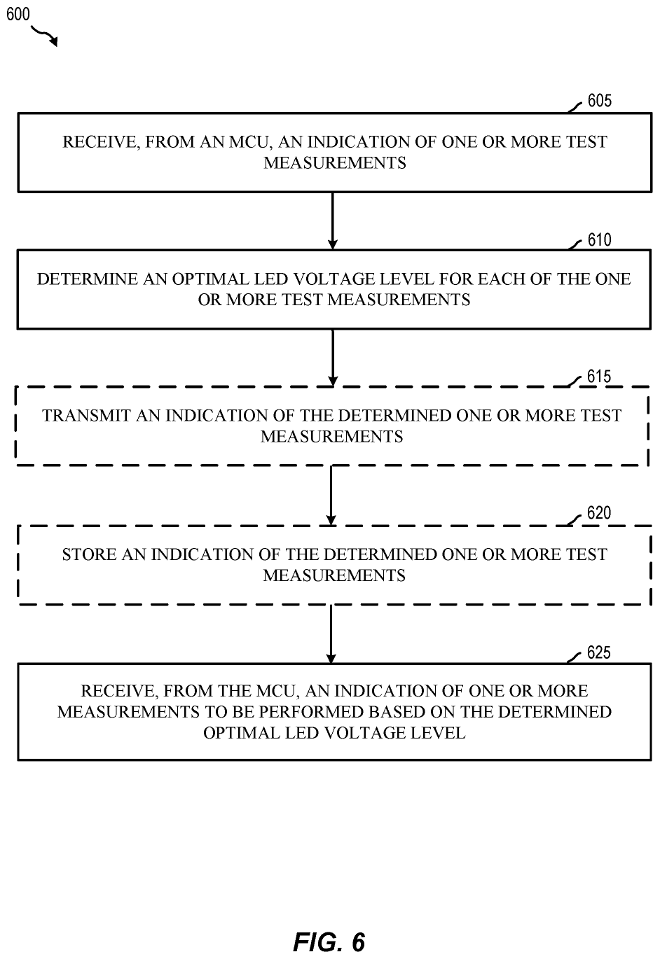

FIG. 6 is a flow diagram illustrating an example learning process 600 for performing the fast DVS function (e.g., as described in relation to FIG. 5) at a PPG device (e.g., PPG device 400 of FIG. 4). The learning process 600 may be performed to determine an optimal voltage level (e.g., optimal V_{F} as described in FIG. 2) for powering each LED of the PPG device 400 during a measurement. Here, the optimal voltage level may be a minimum voltage level required to power a particular LED without voltage dropout at the current sink (e.g., current sink 212 of FIG. 2). The learning process 600 may be performed to determine and store the optimal voltage levels used by the buck boost (e.g., buck boost 402 of FIG. 4) to power the LEDs.

Initially, at 605, the AFE (e.g., AFE 404 of FIG. 4) may receive, from the MCU (e.g., MCU 406 of FIG. 4), an indication of one or more test measurements. In relation to FIG. 5, the one or more test measurements may include the first three measurements (e.g., LED 1, LED 2, LED 3) of the first frame, and the second three measurements (e.g., LED 1, LED 2, LED 1) of the second frame. Thus, the indication of one or more test measurements may indicate which LED(s) to power on at a particular time within a particular frame.

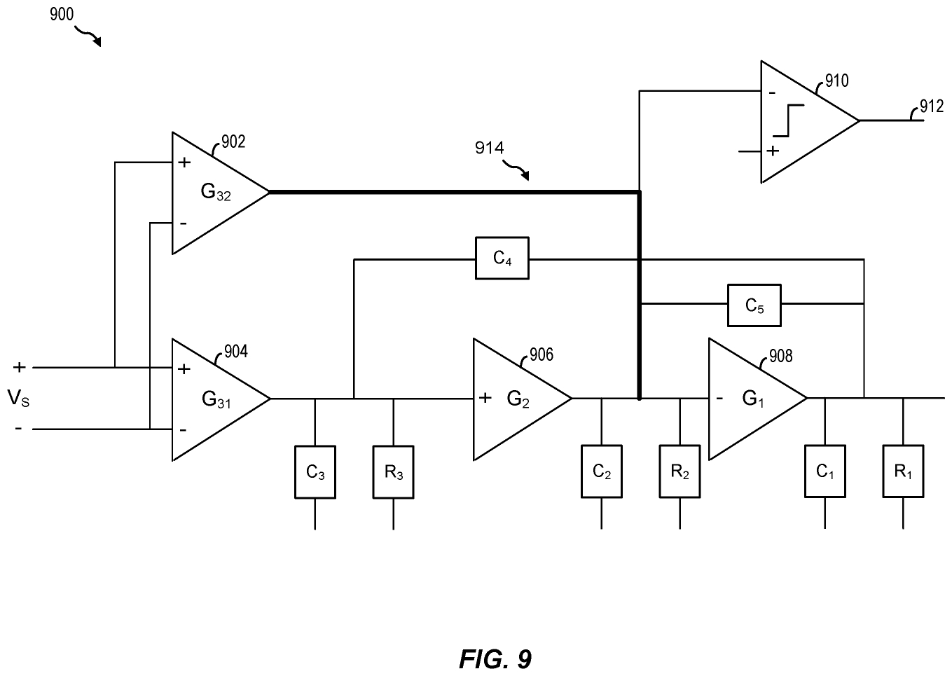

At 610, the AFE 404 may determine an optimal LED voltage level for each of the one or more test measurements by performing a step voltage search for each LED of the one or more test measurements indicated by the MCU 406. Here, the AFE 404 may transmit commands instructing the buck boost 402 to adjust its output voltage by dropping the output voltage incrementally for a particular one or more LEDs of an associated test measurement until the AFE 404 detects a voltage condition (e.g., voltage dropout) across the current sink and associated with one or more LEDs. In this example, the AFE 404 may include a drop-out voltage detector (e.g., as illustrated in FIG. 9).

For example, the one or more test measurements indicated by the MCU 406 may include a first test measurement of a green LED (e.g., LED 1). To perform the first test measurement, the AFE 404 may direct the buck boost 402 to apply an initial voltage level to power-on the green LED. The AFE 404 may then direct the buck boost 402 to reduce the initial voltage level by decremental voltage drops until the AFE 404 detects a dropout condition associated with the green LED at the current sink. In response to detecting the dropout condition, the AFE 404 may trigger an interrupt and increase the voltage across the green LED to an optimal voltage level by increasing the dropout voltage by one or more incremental steps to provide some margin between the dropout voltage and the optimal voltage. Thus, if the dropout condition occurs at a first voltage level, the AFE 404 may increase the first voltage level by one or more incremental values to a second voltage level (e.g., the optimal voltage level).

In certain aspects, the AFE 404 may determine an optimal LED voltage level for each of the one or more test measurements by performing a binary voltage search for each LED of the one or more test measurements indicated by the MCU 406. Here, the AFE 404 may transmit commands instructing the buck boost 402 to adjust its output voltage to a midpoint voltage value of a given voltage range. For example, the binary search works by repeatedly dividing voltage range in half and applying the middle voltage value to see if that value triggers a voltage condition. If the voltage value does not trigger the voltage condition, then the search continues without voltage values greater than the middle voltage value. If the voltage value does trigger the voltage condition, then the search may end (e.g., the optimum voltage value is equal to the middle voltage value) or the search may continue without voltage values that are less than the middle voltage value. The voltage range may be a pre-configured range of voltages stored at the AFE 404 and/or MCU 406. For example, the range of voltage values may be from 0.5V to 5V.

In some examples, the binary search may be continued to determine a maximum voltage value that still triggers the voltage condition. Once this maximum value is found, a pre-configured value may be added to the maximum voltage to determine the optimum voltage for a particular LED. In another example, the search may be continued to determine a lowest voltage that does not trigger the voltage condition. This lowest voltage may then be set as the optimum voltage. Accordingly, the binary search may continue until the optimum voltage is found. In some examples, the AFE 404 may perform the binary search, direct the buck boost 402 to apply the voltage values to a corresponding LED, and detect whether a voltage value triggers the voltage condition (e.g., voltage dropout) across the current sink.

At 615, the AFE 404 may optionally transmit an indication of the determined one or more test measurements (e.g., optimal voltage level(s)) and the corresponding LED(s) to the MCU 406. Using the example above, the AFE 404 may transmit an indication of the second voltage level and the corresponding green LED. The MCU 406 may receive the indication and may store the one or more test measurements and the corresponding LED(s) into a lookup table. For example, the look-up table may be indexed by the corresponding LED. Thus, if the optimal voltage is determined for the green LED during the binary or step voltage search, then the MCU 406 may store the optimal voltage associated with the green LED into a look up table so that the next time the green LED is used for a measurement, the MCU 406 can configure the AFE 404 to run the buck boost 402 at the optimal voltage level.

At 620, the AFE 404 may optionally store an indication of the determined one or more test measurements locally on the AFE 404. For example, the AFE 404 may store one or more of the dropout voltages and/or the optimal voltage on a look-up table indexed by the corresponding LED (e.g., the green LED). Thus, if the AFE 404 performs a subsequent measurement using a green LED, the AFE 404 may refer back to the stored look-up table to determine the optimal voltage level to apply to the green LED via the buck boost 402.

It should be noted, that for either of 615 and 620, the look up table may store currents and voltages for each LED so that the AFE 404 can command the buck boost 402 to use certain currents and/or voltages during a particular measurement. The lookup table can be stored on the AFE 404 (e.g., using a control register), the MCU 406 (e.g., in a memory location), or both. If stored on the AFE 404, the voltage level stored on the control register may be used by the AFE 404 when a subsequent measurement is performed using the corresponding LED.

At 625, the AFE 404 may receive, from the MCU 406, an indication of one or more measurements to be performed based on the determined optimal LED voltage level. Here, the MCU 406 may schedule the AFE 404 to perform one or more measurements using LEDs in each of one or more frames. The AFE 404 may control the buck boost 402 to power the LEDs using the optional LED voltage levels measured during the test.

Referring back to FIG. 4, the AFE 404 may control the buck boost 402 by way of a multi-wire serial interface 408 (e.g., a serial peripheral interface (SPI) or pseudo SPI (P-SPI)) configured to communicatively couple the AFE 404 and buck boost 402. For example, a first wire may be configured to provide a clock signal from the AFE 404 to the buck boost 402 for synchronization, while a second wire may be configured to provide a data signal (e.g., one or more of command signaling, mode signaling, voltage signaling, parity, etc.) to the buck boost 402. The AFE 404 and MCU 406 may communicate by way of a multi-wire interface 410 (e.g., an SPI, inter-integrated circuit (I2C), or other suitable interface). As used herein, a P-SPI may relate to an interface having fewer pins than an SPI. For example, an SPI may include a chip select (CSb) input pin and a master in, slave out (MISO) pin, whereas the P-SPI may omit one or more of these pins, and/or other pins.

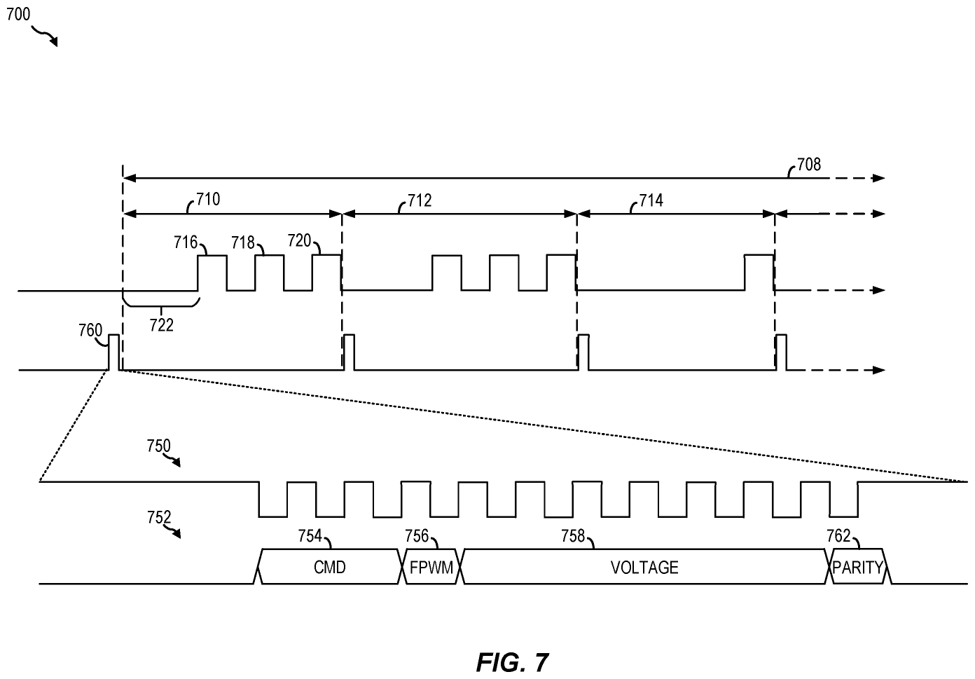

FIG. 7 is a digital signal diagram 700 illustrating example signaling between the AFE 404 and buck boost 402 of a PPG device 400. In some examples, the digital signal diagram 700 illustrates measurements performed after completion of a learning sequence (e.g., as described above in reference to FIG. 6). In such an example, the AFE 404 may direct the buck boost 402 to power the LEDs for each measurement using the optimal LED voltage levels determined during the learning sequence.

Initially, the AFE 404 may transmit a command signal 760 to the buck boost 402, commanding the buck boost 402 to perform an LED measurement and indicating a voltage to be used for a next measurement. The command signal 760 may trigger a first measurement period 710 of one or more measurement periods within a frame 708. As illustrated, the frame 708 may include at least three measurement periods (e.g., the first measurement period 710, a second measurement period 712, and a third measurement period 714). Within each measurement period, the PPG device 400 may perform one or more measurements, with each measurement being an individual firing of an LED within a given measurement period or frame. In one example, the buck boost 402 may power on one LED per measurement period, with multiple measurement periods occurring within a frame (e.g., up to 20 measurement periods within a frame).

The command signal 760 may be transmitted to the buck boost 402 via the multi-wire serial interface 408. In the illustrated example, the command signal 760 includes: a clock signal 750 transmitted via a first wire of the multi-wire serial interface 408, and a command line 752 transmitted via a second wire of the multi-wire serial interface 408. The clock signal 750 may operate at 10 k MHz or any other suitable clock speed and may provide a means for synchronizing operations between the AFE 404 and buck boost 402. The command line 752 may include a command field 754 (e.g., 3-bit) indicative of a buck boost 402 operating mode, a voltage to be used for a particular LED during a measurement period, and/or a shutdown command. The command line 752 may also include 1-bit forced pulse-width modulation (FPWM) mode field 756 configured to indicate whether the buck boost 402 should use an FPWM mode based on whether an LED voltage for an upcoming measurement period is transitioning up or down relative to a previous measurement period. For example, the buck boost 402 may enter into an FPWM mode for transitions from a relatively high voltage level at a first measurement period 710 to a relatively low voltage level at a second measurement period 712. The command line 752 may also include a voltage command field 758 configured to provide the buck boost with one or more voltage level(s) for each LED/ambient light sensor used within a measurement period. A parity bit field 762 may also be included to reduce errors. It should be noted that in some examples, the command line 752 may include fewer than all of the fields illustrated in FIG. 7. In some examples, the command line 752 may be limited to the 1-bit FPWM mode field 756 and the voltage command field 758.

In certain aspects, the command signal 760 may be configured to cause the buck boost 402 to perform all of the measurements within the frame 708. That is, the command signal 760 may provide the buck boost 402 with enough information to allow the buck boost 402 to perform a first measurement 716, a second measurement 718, and a third measurement 720 within the first measurement period 710, and any additional measurements within the second measurement period 712, the third measurement period 714, and any additional measurements periods. Alternatively, the AFE 404 may transmit a command signal 760 at the start of each measurement period. In this case, the command signal 760 may include only enough information to allow the buck boost 402 to perform the first measurement 716, the second measurement 718, and the third measurement 720 within the first measurement period 710. The AFE 404 may provide an additional command signal 760 at the beginning of each subsequent measurement period provide the information necessary for the buck boost 402 to perform measurements within that period.

In some examples, the buck boost 402 may allow a time duration 722 to pass between receiving a command signal from the AFE 404 and initiating measurement(s) in a given measurement period. For example, the first measurement period 710 may begin after the buck boost 402 receives the command signal 760. However, the buck boost 402 may allow the time duration 722 to pass before beginning a first measurement 716 (e.g., powering one or more of an LED/ambient light sensor). This time duration 722 may be configured to allow the buck boost 402 to adjust the LED voltage (e.g., V_{DVS′}) between measurements while reducing any noise that might occur due to the voltage adjustment. For example, the time duration 722 may further provide the buck boost 402 with time to transition from one LED voltage to another LED voltage. For instance, as illustrated in the first frame (FRAME 1) of FIG. 5, the first measurement using LED 1 has a V_{DVS′} representing a lower voltage relative to the second measurement using LED 2 and the third measurement using LED 3. Thus, the time duration 722 may be configured to provide the buck boost 402 with enough time to rapidly transition from: (i) the V_{DVS′} needed for the first measurement, to (ii) the relatively higher V_{DVS} needed for the second measurement. The time duration 722 may be a configurable duration determined by the AFE 404 and/or the MCU 406.

Accordingly, in operation, the AFE 404 may transmit the command signal 760 to the buck boost 402, thereby initiating the first measurement period 710. After the time duration 722 expires, the buck boost 402 may perform a first measurement 716, a second measurement 718, and a third measurement 720, all within the first measurement period 710. In one example, the first measurement 716 and the third measurement 720 may include powering a photodiode 218 as part of an ambient light sensing operation, whereas the second measurement 718 may include powering an LED.

It should be noted that in certain aspects, the PPG device 400 may be configured with a legacy AFE that may not use a digital interface and/or learning algorithm such as the learning algorithm described in reference to FIG. 6. In such an example, instead of the MCU 406 loading the AFE 404 with an indication of an LED voltage level that corresponds to a given measurement period, the MCU 406 may load one or more LED voltage levels into the buck boost 402. Here, the buck boost 402 may store the voltage levels in a look-up table. The AFE 404 can then signal the end of a particular measurement to the buck boost 402 in order to cause the buck boost 402 move on to the next measurement.

In this example, the MCU 406 may store one or more tables of optimal LED voltages. The order of the LED voltages in each table may be aligned with the measurements performed in a particular frame. Thus, each table may correspond to a different frame, wherein each frame uses a different order of LED measurements.

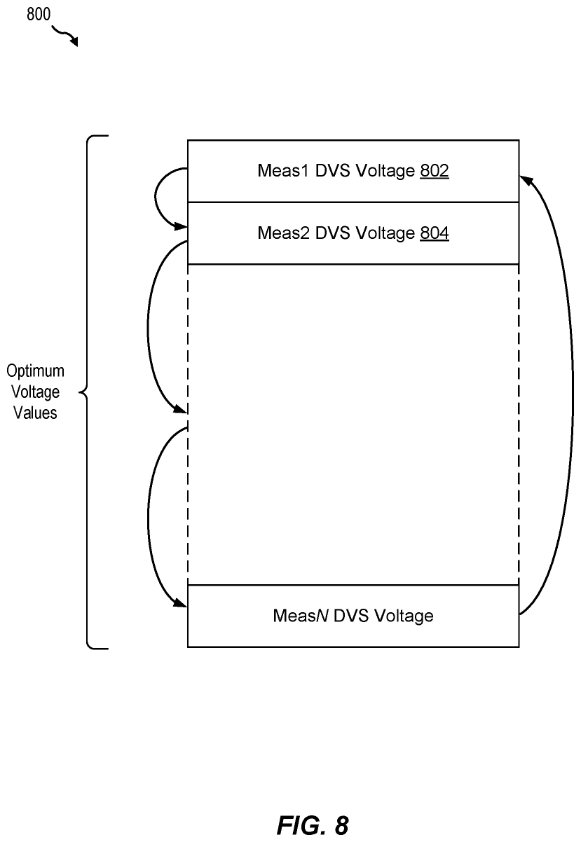

For example, FIG. 8 is a block diagram illustrating an example look-up table 800 that includes multiple rows, each corresponding to an optimal voltage for a particular measurement. In this example, if the AFE 404 is not configured to perform the learning algorithm, the table may be generated by a user and/or a MCU 406 configured with the learning algorithm. As illustrated, the look-up table 800 includes N rows; however, it should be noted that the look-up table 800 may include any suitable number of rows, including 1.

Initially, the MCU 406 may load the look-up table 800 to the buck boost 402 via an interface (e.g., via the AFE 404 or via an I2C link between the MCU 406 and the buck boost 402). The AFE 404 may cause the buck boost 402 to perform the first measurement using a first voltage-level entry 802 of the table by triggering an interrupt. For example, the AFE 404 may communicate an interrupt to the buck boost 402 via one or more interrupt pins. The AFE 404 may cause the buck boost 402 to proceed to the second measurement using a second voltage-level entry 804 by triggering another interrupt.

Once all N measurements of the look-up table 800 have been performed, the MCU 406 may load another table to the buck boost 402. In some examples, the buck boost 402 may cycle through the look-up table 800 in whole or in part. For example, once each of the N measurements of the table are completed, the buck boost 402 may restart the table by performing the first measurement again after completion of the Nth measurement.

In some examples, the buck boost may be configured with a timeout tracker in case the buck boost 402 gets out of synchronization with the AFE 404 and/or MCU 406. Here, the buck boost 402 may be (pre-) configured with a limit on the amount of time a measurement is allowed to be performed. If the measurement exceeds this time limit, the buck boost may terminate or interrupt the measurement to prevent it from consuming excessive resources or causing delays. Upon terminating the measurement, the buck boost may begin performing the next measurement.

FIG. 9 is a simplified schematic illustrating a multipath nested Miller compensation (MNMC) op-amp 900 that may be used to detect voltage dropout across the current sink (e.g., current sink 212 of FIG. 2). The MNMC op-amp 900 includes multiple amplifying elements (e.g., a first amplifying element 902, a second amplifying element 904, a third amplifying element 906, a fourth amplifying element 908, and a fifth amplifying element 910) that may be implemented using transistors. The MNMC op-amp 900 also includes multiple capacitors (e.g., illustrated as C₁₋₅, used for compensation, feedback, and stabilization in the MNMC op-amp 900) and resistors (e.g., illustrated as R₁₋₃, used to control the gain and other parameters of the MNMC op-amp 900). The output 912 of the fifth amplifying element may be coupled to V_{SS} of the op-amp 220 of the AFE 204 of FIG. 2 and may be configured to indicate a voltage condition (e.g., dropout event). It should be noted that the illustrated MNMC op-amp 900 is an example and may be implemented using any suitable number of amplifying elements, capacitors and resistors. The MNMC op-amp 900 may be powered by a voltage source (Vs).

The second amplifying element 904 may function as a main input stage, and the first amplifying element 902 may operate as an input feed-forward stage. The main input stage outputs to an independent parallel path (e.g., illustrated as a bold line, N_{gate} 914) across the main input stage and an intermediate stage (e.g., formed by the third amplifying element 906). The combined output of the first amplifying element 902 and the sum of the second amplifying element 904 and the third amplifying element (e.g., N_{gate} 914) may be used to indicate when a voltage dropout event occurs. For example, in the event of a voltage dropout, the N_{gate} 914 feedback loop forces the voltage output of the AFE op-amp 220 to go nonlinearly higher to compensate for the loss of voltage (e.g., drain-to-source voltage) at the MOSFET 222.

In relation to FIG. 6, the AFE may utilize the MNMC op-amp 900 to determine an optimal LED voltage level for each of the one or more test measurements of a frame at 610. Specifically, the AFE may gradually reduce an initial voltage applied across an LED until a voltage dropout event is detected at the AFE via the MNMC op-amp 900.

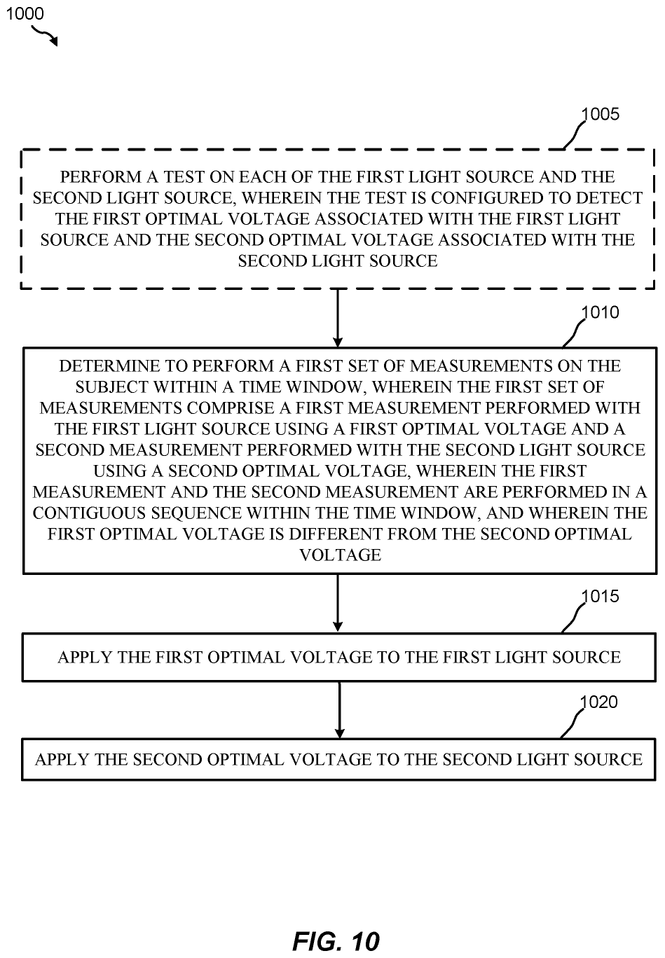

FIG. 10 is a simplified flow diagram illustrating example operations 1000 that may be performed by the PPG device 400 of FIG. 4. Initially, the example operations 1000 may optionally include a first function 1005, wherein the PPG device 400 may perform a test on each of the first light source and the second light source, wherein the test is configured to detect the first optimal voltage associated with the first light source and the second optimal voltage associated with the second light source. In some examples, the AFE 204/404 may perform a test on each light source to determine an optimum voltage for powering the light source. The test may include the AFE 204/404 applying an initial voltage across the first light source (e.g., green LED). The initial voltage may be a relatively high voltage (e.g., V_{DVS} of FIG. 5) that is pre-configured to successfully power the first light source.

From there, the AFE 204/404 may gradually reduce the voltage across the first light source. For example, the AFE 204/404 may reduce the voltage by pre-configured increments (e.g., 0.2V) until a voltage dropout condition is detected at the AFE 204/404 via the MNMC op-amp 900 of FIG. 9. Once the dropout condition is detected by the AFE 204/404, the AFE may proceed to determine the optimum voltage by adding a preconfigured voltage to the voltage level applied to the first light source when the dropout condition occurred. For example, if the voltage level at dropout is 2V, then the AFE 204/404 may add the preconfigured voltage (e.g., 0.2V) to that voltage level, thereby making the optimum voltage level of the first light source 2.2V. The AFE 204/404 may proceed to perform the same test on additional light sources of the PPG device 400. Accordingly, an optimum voltage may be learned for each light source so that a minimum operating voltage (e.g., V_{DVS′} of FIG. 5) may be applied during a measurement associated with each light source within a frame as illustrated in FIG. 5.

At a second function 1010, the PPG device 400 may determine to perform a first set of measurements on the subject within a time window, wherein the first set of measurements comprise a first measurement performed with the first light source using a first optimal voltage and a second measurement performed with the second light source using a second optimal voltage, wherein the first measurement and the second measurement are performed in a contiguous sequence within the time window, and wherein the first optimal voltage is different from the second optimal voltage. In some examples, a gap of time may exist between the first measurement and the second measurement wherein the buck boost does not apply a voltage across any of the light sources. In some examples, such a gap of time may be within 2-25 μs. Here, the time window may relate to a frame as illustrated in FIG. 5, and the first set of measurements may include applying optimal voltages to corresponding light sources associated with the measurements within that frame. As illustrated in FIG. 5, three measurements are performed within FRAME 1 in a contiguous sequence (e.g., one after the other).

At a third function 1015, the PPG device 400 may apply the first optimal voltage to the first light source. For example, after the AFE 204/404 is configured by the MCU 406 to perform a measurement, the AFE 204/404 may apply an optimum voltage across a light source associated with that measurement. For example, referring to FIG. 5, the AFE may apply the first optimal voltage (e.g., V_{DVS′}) to LED 1 of FRAME 1.

At a fourth function 1020, the PPG device may apply the second optimal voltage to the second light source. Here, based on the same configuration from the MCU 406, the AFE 204/404 may apply another optimum voltage across another light source associated with another measurement. For example, referring to FIG. 5, the AFE may apply the second optimal voltage (e.g., V_{DVS′}) to LED 2 of FRAME 1.

In certain aspects, the AFE 204/404 may perform a test (e.g., 610 of FIG. 6) to determine an optimum voltage of an LED while performing a measurement. For example, while performing a measurement via a given LED (e.g., at the third function 1015 and/or the fourth function 1020), the AFE 204/404 may detect a voltage condition at the current sink (e.g., if the current sink voltage has gone out of range). This could occur due to LED forward voltage drift due to age and/or temperature, or implementation error. Thus, if the first optimal voltage is not an ideal voltage to apply to the first light source, the AFE 204/404 may detect a voltage condition at the current sink. In response to detecting the voltage condition, the AFE 204/404 may perform one of the step voltage search or the binary voltage search described above in connection with FIG. 6 to determine a new optimum voltage for the first light source. Once determined, the AFE 204/404 may continue the first measurement by causing the buck boost to apply the new optimum voltage to the first light source. The AFE 204/404 may store the new optimum voltage value for future measurements with the first light source. Accordingly, the AFE 204/404 may tune and update optimum voltages associated with different light sources during runtime of the PPG device.

In certain aspects, the first optimal voltage is applied to the first light source at a first time instance, wherein the second optimal voltage is applied to the second light source at a second time instance, and wherein a delay between the first time instance and the second time instance is equal to or less than 1 millisecond. In other words, there may be a delay between measurements. Referring back to FIG. 5, a time gap is illustrated between each measurement (e.g., LED 1, LED 2, LED 3) within a frame. That time gap may be equal to or less than a millisecond, and may be within a time window having a duration of 2 μs-24 μs. In contrast, each measurement may have a duration between 10 μs
120 μs.

In certain aspects, the test is performed prior to performance of the first set of measurements and in response to the determination to perform the first set of measurements. For example, the MCU may provide the AFE with a command to perform a set of measurements within a frame. In response to the command, the AFE may perform the test to determine optimum voltages associated with each light source to be used in the set of measurements. The AFE may then perform the set of measurements using the determined optimum voltages, for examples, as illustrated in FIG. 5.

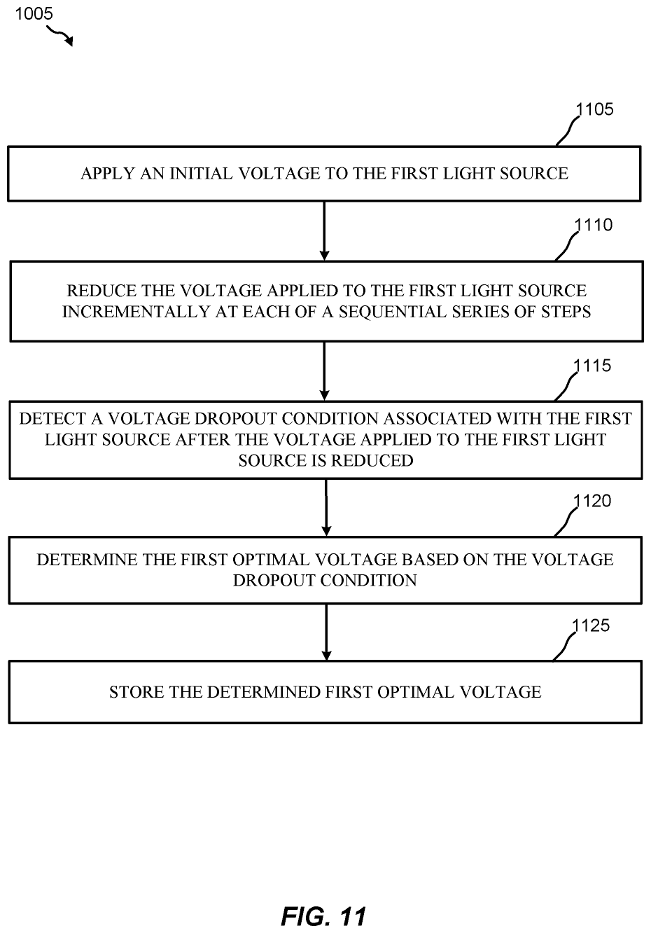

FIG. 11 is a simplified flow diagram illustrating example operations associated with the first function 1005 of FIG. 10. At a first function 1105, the PPG device 400 may apply an initial voltage to the first light source. The initial voltage may be a relatively high voltage (e.g., V_{DVS} of FIG. 5 relative to V_{DVS′}) that is pre-configured to successfully power all of the light sources of the PPG device 400.

At a second function 1110, the AFE may reduce the voltage applied to the first light source incrementally at each of a sequential series of steps. For example, the AFE may gradually reduce the voltage applied across the first light source from the initial voltage level. The voltage reduction may be performed in steps (e.g., increments between 0.001V-0.5V) where each reduced level of voltage is held for a pre-configured amount of time suitable to allow the AFE to detect a dropout condition.

At a third function 1115, the AFE may detect a voltage dropout condition associated with the first light source after the voltage applied to the first light source is reduced. For example, if the first light source is a green LED and the initial voltage is 5V, then the AFE may incrementally drop the voltage across the first light source by 0.2V at periodic intervals (e.g., 15 intervals in this example). The AFE may detect a dropout condition at 2.0V, wherein the green LED fails and a dropout occurs that the current sink 212.

At a fourth function 1120, the AFE may determine the first optimal voltage based on the voltage dropout condition. Here, the AFE may determine that the dropout condition for the green LED occurred after reducing the initial voltage to 2.0V. Accordingly, the AFE may determine an optimal voltage by adding a pre-configured (e.g., by the MCU) value to the dropout voltage. Thus, if the preconfigured value is 0.2V, then the AFE may determine that the optimum voltage for the green LED is 2.2V. In some examples, the preconfigured value may be the same as the incremental value (e.g., 0.2V). Alternatively, the preconfigured value may correspond to a different value defined by the MCU.

At a fifth function 1125, the AFE may store the determined first optimal voltage. Here, the AFE may store the first optimal voltage in a register that corresponds to the first light source, or in a location in the register that corresponds to the first light source. In some examples, the AFE may provide an indication of the determined first optimal voltage level to the MCU, and the MCU may store the value in a memory.

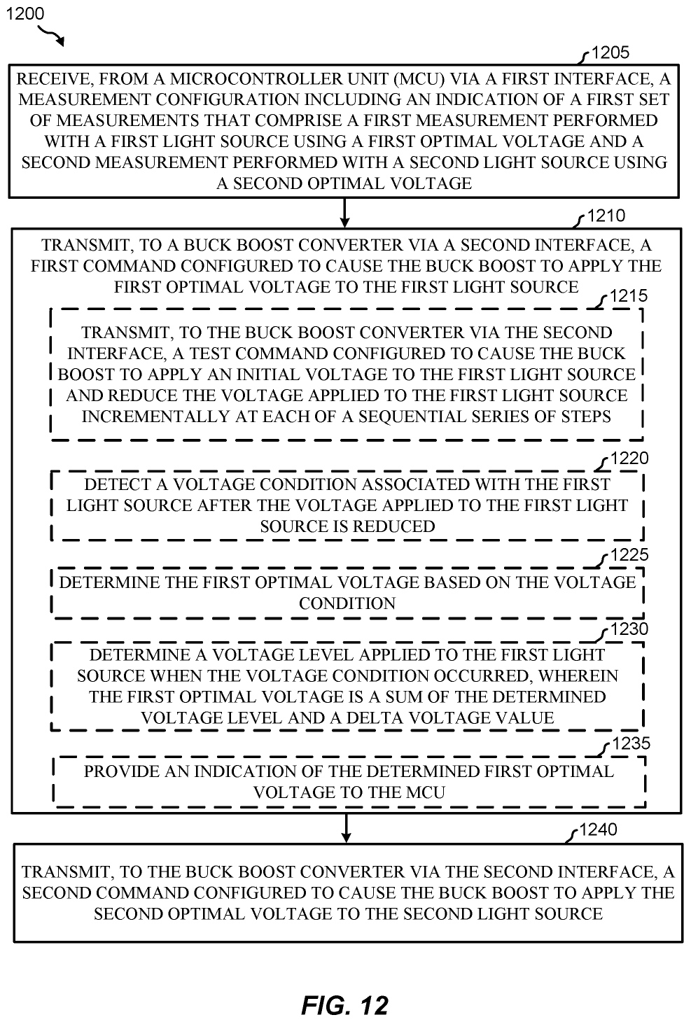

FIG. 12 is a simplified flow diagram illustrating example operations 1200 that may be performed by the PPG device 400 of FIG. 4. The example operations 1200 Initially, the example operations 1200 may include a first function 1205, wherein the PPG device 400 may receive, from a microcontroller unit (MCU) via a first interface, a measurement configuration including an indication of a first set of measurements that comprise a first measurement performed with a first light source using a first optimal voltage and a second measurement performed with a second light source using a second optimal voltage. For examples, the MCU provide commands to the AFE to perform certain measurements (e.g., PPG measurements on a subject).

The example operations 1200 may include a second function 1210, wherein the PPG device 400 may transmit, to a buck boost converter via a second interface, a first command configured to cause the buck boost to apply the first optimal voltage to the first light source. Here, the AFE may cause the buck boost to apply a known voltage to the first light source based on the measurement configuration received from the MCU.

The example operations 1200 may optionally include a third function 1215, wherein the PPG device 400 may transmit, to the buck boost converter via the second interface, a test command configured to cause the buck boost to apply an initial voltage to the first light source and reduce the voltage applied to the first light source incrementally at each of a sequential series of steps. Here, the AFE may determine that the first optimal voltage is not sufficient to power the first light source. As such, the AFE may determine to perform a sequential test to determine a new optimal voltage. Once the new optimal voltage is determined, the AFE may perform the first measurement using the new optimal voltage.

The example operations 1200 may optionally include a fourth function 1220, wherein the PPG device 400 may detect a voltage condition associated with the first light source after the voltage applied to the first light source is reduced. Here, as part of the test described in the third function 1215, the AFE may gradually reduce the voltage applied to the first light source to determine the new optimal voltage.

The example operations 1200 may optionally include a fifth function 1225, wherein the PPG device 400 may determine the first optimal voltage based on the voltage condition. Here, the voltage condition may indicate that the LED cannot be powered with the level of voltage applied by the AFE and buck boost. Thus, when the voltage condition is detected, the AFE may determine the new optimal voltage by adding a preconfigured value to the level of applied voltage.

The example operations 1200 may optionally include a sixth function 1230, wherein the PPG device 400 may determine a voltage level applied to the first light source when the voltage condition occurred, wherein the first optimal voltage is a sum of the determined voltage level and a delta voltage value. Here, the delta voltage level may relate to the preconfigured value discussed in connection with the fifth function 1225.

The example operations 1200 may optionally include a seventh function 1235, wherein the PPG device 400 may provide an indication of the determined first optimal voltage to the MCU. Here, the AFE may provide the MCU with an indication of the new optimal voltage for storage. This way, the MCU may include the new optimal voltage in future commands.

The example operations 1200 may include an eighth function 1240, wherein the PPG device 400 may transmit, to the buck boost converter via the second interface, a second command configured to cause the buck boost to apply the second optimal voltage to the second light source. Here, the AFE may trigger performance of the second measurement after completion of the first measurement.

In certain aspects, the first command comprises an indication of the first light source and the first optimal voltage, and wherein the second command comprises an indication of the second light source and the second optimal voltage.

In certain aspects, the first command further comprises a forced pulse-width modulation (FPWM) mode field indicating whether the buck boost will use an FPWM mode to transition the first optimal voltage to the second optimal voltage.

In certain aspects, the AFE is directly coupled to the MCU via the first interface, and wherein the AFE is directly coupled to the buck boost converter via the second interface.

### Example Aspects

Example 1 is a method for capturing measurements related to health of a subject, comprising: determining to perform a first set of measurements on the subject within a time window, wherein the first set of measurements comprise a first measurement performed with a first light source using a first optimal voltage and a second measurement performed with a second light source using a second optimal voltage, wherein the first measurement and the second measurement are performed in a contiguous sequence within the time window, and wherein the first optimal voltage is different from the second optimal voltage; applying the first optimal voltage to the first light source; and applying the second optimal voltage to the second light source.

Example 2 is the method of example 1, wherein the first optimal voltage is applied to the first light source at a first time instance, wherein the second optimal voltage is applied to the second light source at a second time instance, and wherein a delay between the first time instance and the second time instance is equal to or less than 1 millisecond.

Example 3 is the method of any of examples 1 and 2, further comprising: performing a test on each of the first light source and the second light source, wherein the test is configured to detect the first optimal voltage associated with the first light source and the second optimal voltage associated with the second light source.

Example 4 is the method of example 3, wherein the method further comprises: applying an initial voltage to the first light source; reducing the voltage applied to the first light source incrementally at each of a sequential series of steps; detecting a voltage condition associated with the first light source after the voltage applied to the first light source is reduced; and determining the first optimal voltage based on the voltage condition.

Example 5 is the method of example 4, further comprising: storing the determined first optimal voltage.

Example 6 is the method of any of examples 3-5, wherein the test comprises a binary search of a range of voltage values.

Example 7 is the method of any of examples 3-6, wherein the test is performed prior to performance of the first set of measurements and in response to the determination to perform the first set of measurements.

Example 8 is the method of any of examples 1-7, further comprising: detecting a voltage condition associated with the first light source upon application of the first optimal voltage to the first light source; performing a test on the first light source in response to detection of the voltage condition, wherein the test is configured to detect a third optimal voltage associated with the first light source; and applying the third optimal voltage to the first light source.

Example 9 is a method for capturing measurements related to health of a subject at an analog front end (AFE), comprising: receiving, from a microcontroller unit (MCU) via a first interface, a measurement configuration including an indication of a first set of measurements that comprise a first measurement performed with a first light source using a first optimal voltage and a second measurement performed with a second light source using a second optimal voltage; transmit, to a buck boost converter via a second interface, a first command configured to cause the buck boost to apply the first optimal voltage to the first light source; and transmit, to the buck boost converter via the second interface, a second command configured to cause the buck boost to apply the second optimal voltage to the second light source.

Example 10 is the method of example 9, wherein the first command comprises an indication of the first light source and the first optimal voltage, and wherein the second command comprises an indication of the second light source and the second optimal voltage.

Example 11 is the method of example 10, wherein the first command further comprises a forced pulse-width modulation (FPWM) mode field indicating whether the buck boost will use an FPWM mode to transition the first optimal voltage to the second optimal voltage.

Example 12 is the method of any of examples 9-11, wherein the AFE is directly coupled to the MCU via the first interface, and wherein the AFE is directly coupled to the buck boost converter via the second interface.

Example 13 is the method of any of examples 9-12, further comprising: transmitting, to the buck boost converter via the second interface, a test command configured to cause the buck boost to apply an initial voltage to the first light source and reduce the voltage applied to the first light source incrementally at each of a sequential series of steps; detecting a voltage condition associated with the first light source after the voltage applied to the first light source is reduced; and determining the first optimal voltage based on the voltage condition.

Example 14 is the method of example 13, further comprising: transmitting the test command in response to receiving the measurement configuration.

Example 15 is the method of any of examples 13 and 14, wherein determining the first optimal voltage comprises: determining a voltage level applied to the first light source when the voltage condition occurred, wherein the first optimal voltage is a sum of the determined voltage level and a delta voltage value.

Example 16 is the method of any of examples 13-15, further comprising: providing an indication of the determined first optimal voltage to the MCU.

Example 17 is an apparatus for capturing measurements related to health of a subject, comprising means for performing a method in accordance with any one of examples 1-8.

Example 18 is an apparatus for capturing measurements related to health of a subject at an analog front end (AFE), comprising means for performing a method in accordance with any one of examples 9-16.

Example 19 is a non-transitory computer-readable medium comprising instructions that, when executed by an apparatus, cause the apparatus to perform a method in accordance with any one of examples 1-8.

Example 20 is a non-transitory computer-readable medium comprising instructions that, when executed by an apparatus, cause the apparatus to perform a method in accordance with any one of examples 9-16.

Example 21 is an apparatus for capturing measurements related to health of a subject, comprising: one or more memories, individually or in combination, having instructions; and one or more processors, individually or in combination, configured to execute the instructions and cause the apparatus to perform a method in accordance with any one of examples 1-8.

Example 22 is an apparatus for capturing measurements related to health of a subject, comprising: one or more memories, individually or in combination, having instructions; and one or more processors, individually or in combination, configured to execute the instructions and cause the apparatus to perform a method in accordance with any one of examples 9-16.

### ADDITIONAL CONSIDERATIONS

The above detailed description set forth above in connection with the appended drawings describes examples and does not represent the only examples that may be implemented or that are within the scope of the claims. The term “example,” when used in this description, means “serving as an example, instance, or illustration,” and not “preferred” or “advantageous over other examples.” The detailed description includes specific details for the purpose of providing an understanding of the described techniques. These techniques, however, may be practiced without these specific details. For example, changes may be made in the function and arrangement of elements discussed without departing from the scope of the disclosure. Also, various examples may omit, substitute, or add various procedures or components as appropriate. For instance, the methods described may be performed in an order different from that described, and various steps may be added, omitted, or combined. Also, features described with respect to some examples may be combined in other examples. In some instances, well-known structures and apparatuses are shown in block diagram form in order to avoid obscuring the concepts of the described examples.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, computer-executable code or instructions stored on a computer-readable medium, or any combination thereof.

Several aspects of photoplethysmography (PPG) systems are presented herein with reference to various apparatus and methods. These apparatus and methods are described in the detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example aspects, the functions described may be implemented in hardware, software, or any combination thereof. If implemented in software, the functions may be stored on or encoded as one or more instructions or code on a computer-readable medium. Computer-readable media includes computer storage media. Storage media may be any available media that may be accessed by a computer. By way of example, and not limitation, such computer-readable media may comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that may be used to store computer executable code in the form of instructions or data structures that may be accessed by a computer.

The various illustrative blocks and components described in connection with the disclosure herein may be implemented or performed with a specially-programmed device, such as but not limited to a processor, a digital signal processor (DSP), an ASIC, a FPGA or other programmable logic device, a discrete gate or transistor logic, a discrete hardware component, or any combination thereof designed to perform the functions described herein. A specially-programmed processor may be a microprocessor, but in the alternative, the processor may be any conventional processor, controller, microcontroller, or state machine. A specially-programmed processor may also be implemented as a combination of computing devices, e.g., a combination of a DSP and a microprocessor, multiple microprocessors, one or more microprocessors in conjunction with a DSP core, or any other such configuration.

The functions described herein may be implemented in hardware, software executed by a processor, firmware, or any combination thereof. If implemented in software executed by a processor, the functions may be stored on or transmitted over as one or more instructions or code on a non-transitory computer-readable medium. Other examples and implementations are within the scope and spirit of the disclosure and appended claims. For example, due to the nature of software, functions described above may be implemented using software executed by a specially programmed processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and B and C).

Computer-readable media includes both computer storage media and communication media including any medium that facilitates transfer of a computer program from one place to another. A storage medium may be any available medium that may be accessed by a general purpose or special purpose computer. By way of example, and not limitation, computer-readable media may comprise RAM, ROM, EEPROM, CD-ROM or other optical disk storage, magnetic disk storage or other magnetic storage devices, or any other medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The previous description of the disclosure is provided to enable a person skilled in the art to make or use the disclosure. Various modifications to the disclosure will be readily apparent to those skilled in the art, and the common principles defined herein may be applied to other variations without departing from the spirit or scope of the disclosure. Furthermore, although elements of the described aspects may be described or claimed in the singular, the plural is contemplated unless limitation to the singular is explicitly stated. Additionally, all or a portion of any aspect may be utilized with all or a portion of any other aspect, unless stated otherwise. Thus, the disclosure is not to be limited to the examples and designs described herein but is to be accorded the widest scope consistent with the principles and novel features disclosed herein.

What is claimed is:

1. An apparatus for capturing measurements related to health of a subject, comprising:
a plurality of light sources comprising a first light source and a second light source configured to emit electromagnetic radiation;
one or more memories, individually or in combination, having instructions; and
one or more processors, individually or in combination, configured to execute the instructions and cause the apparatus to:
determine to perform a first set of measurements on the subject within a time window, wherein the first set of measurements comprise a first measurement performed with the first light source using a first optimal voltage and a second measurement performed with the second light source using a second optimal voltage, wherein the first measurement and the second measurement are performed in a contiguous sequence within the time window, and wherein the first optimal voltage is different from the second optimal voltage;
apply the first optimal voltage to the first light source; and
apply the second optimal voltage to the second light source.

2. The apparatus of claim 1, wherein the first optimal voltage is applied to the first light source at a first time instance, wherein the second optimal voltage is applied to the second light source at a second time instance, and wherein a delay between the first time instance and the second time instance is equal to or less than 1 millisecond.

3. The apparatus of claim 1, wherein the one or more processors, individually or in combination, are further configured to cause the apparatus to:
perform a test on each of the first light source and the second light source, wherein the test is configured to detect the first optimal voltage associated with the first light source and the second optimal voltage associated with the second light source.

4. The apparatus of claim 3, wherein the one or more processors, individually or in combination, being configured to cause the apparatus to perform the test on each of the first light source and the second light source, are further configured to cause the apparatus to:
apply an initial voltage to the first light source;
reduce the voltage applied to the first light source incrementally at each of a sequential series of steps;
detect a voltage condition associated with the first light source after the voltage applied to the first light source is reduced; and
determine the first optimal voltage based on the voltage condition.

5. The apparatus of claim 4, wherein the one or more processors, individually or in combination, are further configured to cause the apparatus to:
store the determined first optimal voltage.

6. The apparatus of claim 3, wherein the test comprises a binary search of a range of voltage values.

7. The apparatus of claim 3, wherein the test is performed prior to performance of the first set of measurements and in response to the determination to perform the first set of measurements.

8. The apparatus of claim 1, wherein the one or more processors, individually or in combination, are further configured to cause the apparatus to:
detect a voltage condition associated with the first light source upon application of the first optimal voltage to the first light source;
perform a test on the first light source in response to detection of the voltage condition, wherein the test is configured to detect a third optimal voltage associated with the first light source; and
apply the third optimal voltage to the first light source.

9. A method for capturing measurements related to health of a subject at an analog front end (AFE), comprising:
receiving, from a microcontroller unit (MCU) via a first interface, a measurement configuration including an indication of a first set of measurements that comprise a first measurement performed with a first light source using a first optimal voltage and a second measurement performed with a second light source using a second optimal voltage;
transmitting, to a buck boost converter via a second interface, a first command configured to cause the buck boost to apply the first optimal voltage to the first light source; and
transmitting, to the buck boost converter via the second interface, a second command configured to cause the buck boost to apply the second optimal voltage to the second light source.

10. The method of claim 9, wherein the first command comprises an indication of the first light source and the first optimal voltage, and wherein the second command comprises an indication of the second light source and the second optimal voltage.

11. The method of claim 10, wherein the first command further comprises a forced pulse-width modulation (FPWM) mode field indicating whether the buck boost will use an FPWM mode to transition the first optimal voltage to the second optimal voltage.

12. The method of claim 9, wherein the AFE is directly coupled to the MCU via the first interface, and wherein the AFE is directly coupled to the buck boost converter via the second interface.

13. The method of claim 9, further comprising:
transmitting, to the buck boost converter via the second interface, a test command configured to cause the buck boost to apply an initial voltage to the first light source and reduce the voltage applied to the first light source incrementally at each of a sequential series of steps;
detecting a voltage condition associated with the first light source after the voltage applied to the first light source is reduced; and
determining the first optimal voltage based on the voltage condition.

14. The method of claim 13, wherein the test command is transmitted in response to receiving the measurement configuration.

15. The method of claim 13, wherein determining the first optimal voltage comprises:
determining a voltage level applied to the first light source when the voltage condition occurred, wherein the first optimal voltage is a sum of the determined voltage level and a delta voltage value.

16. The method of claim 13, further comprising:
providing an indication of the determined first optimal voltage to the MCU.

17. A non-transitory, computer-readable medium comprising computer executable code, the code when executed by one or more processors causes the one or more processors to, individually or in combination:
determine to perform a first set of measurements on a subject within a time window, wherein the first set of measurements comprise a first measurement performed with a first light source using a first optimal voltage and a second measurement performed with a second light source using a second optimal voltage, wherein the first measurement and the second measurement are performed in a contiguous sequence within the time window, and wherein the first optimal voltage is different from the second optimal voltage;
apply the first optimal voltage to the first light source; and
apply the second optimal voltage to the second light source.

18. The non-transitory, computer-readable medium of claim 17, wherein the first optimal voltage is applied to the first light source at a first time instance, wherein the second optimal voltage is applied to the second light source at a second time instance, and wherein a delay between the first time instance and the second time instance is equal to or less than 1 millisecond.

19. The non-transitory, computer-readable medium of claim 17, wherein the code when executed by one or more processors further causes the one or more processors to, individually or in combination:
perform a test on each of the first light source and the second light source, wherein the test is configured to detect the first optimal voltage associated with the first light source and the second optimal voltage associated with the second light source.

20. The non-transitory, computer-readable medium of claim 19, wherein the code when executed by one or more processors further causes the one or more processors to, individually or in combination:
apply an initial voltage to the first light source;
reduce the voltage applied to the first light source incrementally at each of a sequential series of steps;
detect a voltage condition associated with the first light source after the voltage applied to the first light source is reduced; and
determine the first optimal voltage based on the voltage condition.
